(19) United States
(12) Patent Application Publication
(10) Pub. No.: US 2026/0066146 A1
(43) Pub. Date: Mar. 5, 2026

(54) TABLE MOUNTED SHIELDING SYSTEM

(71) Applicant: RAMPART IC, Inc., Birmingham, AL (US)

(72) Inventors: William Thomas LIVINGSTON, Vestavia Hills, AL (US); J. Dylan QUICK, Vestavia Hills, AL (US)

(21) Appl. No.: 19/386,039

(22) Filed: Nov. 11, 2025

Related U.S. Application Data
(63) Continuation of application No. PCT/US24/29154, filed on May 13, 2024.
(60) Provisional application No. 63/466,189, filed on May 12, 2023.

(51) Int. Cl. G21F 3/00; A61B 6/04; A61B 6/10

(52) U.S. Cl. CPC G21F 3/00; A61B 6/0407; A61B 6/107

(57) ABSTRACT

A radiation shield assembly is described, configured to block radiation emanating from a radiation source from reaching a user. A first curtain bar includes one or more mounting slots, each mounting slot attached to a respective mounting bracket. The one or more mounting slots include a plurality of furrow pairs of distinct depths. The mounting bracket includes a pair of protrusions fitting into a respective furrow pair on a curtain bar attachment end and a c-shaped aperture on a rail attachment end. A width of the c-shaped aperture is adjusted based on the placement of the protrusions in respective furrow pairs. The first curtain bar is attached to a second curtain bar by a pivoting anti-sag joint. An accessory rail is attached to the first curtain bar that is adjustable between two or more distinct widths. The anti-sag joint includes a convex conical section and a complementary concave conical section.

### CROSS-REFERENCE TO RELATED APPLICATION(S)

This application is a continuation of International Patent Application No. PCT/US2024/029154, filed May 13, 2024, which claims the benefit of U.S. Provisional Ser. No. 63/466,189 , filed May 12, 2023, which are incorporated herein by reference in their entireties.

### TECHNICAL FIELD

The present disclosure relates generally to radiation protection devices, and specifically to a table mounted shielding system to protect medical personnel from radiological hazards in the operating room.

### BACKGROUND

During many medical procedures an X-ray projector is placed below a patient table, and is directed upward so that a live X-ray image of the patient is available throughout the procedure. This can expose the medical personnel to high levels of X-ray radiation throughout their careers. As a result, it is common to place a shield between the X-ray projector and the medical staff.

However, many existing designs have some unsolved problems. One such problem is that different models of patient tables have rails of different dimensions on which shields are designed to attach. Past attempts to make a shield that can be mounted on rails of different dimensions have employed set screws. Set screws are not ideal for this application, because set screws can come loose, and set screws can damage the rail of the patient table.

Another problem is that the table rail is often used to mount other medical devices necessary for medical procedures. If it the table rail is occupied by the shield, then it is not available for another device. Some current models have an additional rail that is intended to mimic and function like the rail on the table. However, because table rails vary dimensionally, and a given facility has medical devices that are configured to attach to rails of certain dimensions, facilities must buy additional rails of the right dimension to fit medical devices to the given rails of a patient table.

Another problem arises when the mounting system uses two or more jointed bars. Usually this involves a first, larger bar that is mounted to the table rail. A second bar (sometimes two) is attached to the first via a hinge-type joint. The bars are quite heavy, and the second bar will sag under its own weight. The sagging cause excessive wear on the joint and an undesirable sagging of the curtain bar with respect to the rail of the patient table.

The disclosed embodiments are directed to these and other considerations.

### SUMMARY

The present disclosure describes a radiation shield assembly that addresses the problems described above by providing an assembly that is configured to mount to a rail of a patient table, although it is to be understood that not every embodiment of the shield assembly will address every such problem. The assembly includes a curtain bar that may be configured to fit a plurality of standard patient rail sizes. The curtain bar can include an accessory rail that enables a user of the assembly to mount additional medical devices to the patient table even when the radiation shield assembly is in place. The accessory rail may be adjustable in width such that medical devices configured to attach to various dimensions of table rails can be attached to the accessory rail. The assembly may include attachment means to allow radiation blocking curtains to be removably attached to the assembly to block radiation emanating from below the patient table. The curtain bar can be attached to one or more secondary curtain bars by a pivoting joint. Each of the one or more secondary curtain bars can include attachment means for additional radiation blocking curtains to block radiation emanating from below the patient table. The pivoting joints can be constructed to prevent the one or more secondary curtain bars from sagging with respect to the rail of the patient table. The pivoting joint can allow the user of the device to rotate the one or more secondary curtain bars with respect to the curtain bar, such that the radiation blocking curtains can be positioned as desired by a user of the assembly.

In a general, a radiation shield assembly is provided, configured to block radiation emanating from a radiation source. In the first general embodiment, the assembly comprises a first curtain bar configured to attach to rail of the table.

In a first exemplary embodiment, the first curtain bar comprises one or more mounting slots. Each of the one or more mounting slots comprises a plurality of furrow pairs of distinct depths. The assembly comprises one or more mounting brackets. Each of the one or more mounting brackets comprises a curtain bar attachment end and a rail attachment end. The curtain bar attachment end is fastened to a respective mounting slot of the one or more mounting slots of the first curtain bar. The curtain bar attachment end comprises a pair of protrusions. The rail attachment end comprises a c-shaped aperture configured to attach to the rail of the table. The pair of protrusions of the curtain bar attachment end can be placed into a respective furrow pair of the plurality of furrow pairs of a respective mounting slot to adjust a width of the c-shape aperture to thereby fit a plurality of distinct rail widths.

In a second exemplary embodiment, the assembly includes an accessory rail attached to the first curtain bar. The accessory rail is adjustable between two or more distinct widths.

In a third exemplary embodiment, the assembly includes a second curtain bar that is pivotably attached to the first curtain bar by a pivoting anti-sag joint. The pivoting anti-sag joint comprises an upper tab formed by an end of the second curtain bar, a lower tab formed by an end of the first curtain bar, a hinge bolt pivotably connecting the upper tab to the lower tab, a convex conical section formed by the end of the first curtain bar, and a complementary concave conical section formed by the end of the second curtain bar. The convex conical section is configured to support the complementary concave section by direct contact, thereby preventing sagging of the second and third curtain bars. The complementary concave conical section is configured to pivot about the convex conical section.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

FIG. 1. An embodiment of the shield assembly showing a first curtain bar, a second curtain bar, a third curtain bar, radiation blocking curtains, and radiation blocking panels.

FIG. 2. The shield assembly of FIG. 1, showing an exploded view of the components.

FIG. 3A. A top perspective view of the shield assembly of FIG. 1, showing the rotation of a second curtain bar about one or more pivotable joints.

FIG. 3B. A front perspective view of the shield assembly of FIG. 1, showing the rotation of a second curtain bar about one or more pivotable joints.

FIG. 4A. A top perspective view of the shield assembly of FIG. 1, showing the rotation of a second curtain bar about one or more pivotable joints, with the radiation blocking panels removed.

FIG. 4B. A front perspective view of the shield assembly of FIG. 1, showing the rotation of a second curtain bar about one or more pivotable joints, with the radiation blocking panels removed.

FIG. 5A. A perspective view of the assembled second curtain bar.

FIG. 5B. An exploded perspective view of the second curtain bar of FIG. 5A.

FIG. 5C. A perspective view of another embodiment of the assembled second curtain bar.

FIG. 5D. An exploded perspective view of the second curtain bar of FIG. 5C.

FIG. 6. A perspective view of the assembled third curtain bar.

FIG. 7A. A perspective view of the assembled first curtain bar.

FIG. 7B. An exploded perspective view of the first curtain bar of FIG. 7A.

FIG. 7C. A perspective view of an accessory rail bracket as shown in FIG. 7A-7B.

FIG. 7D. A detail view of an accessory bolt aperture of the accessory rail bracket.

FIG. 7E. A front view of the first curtain bar of FIG. 7A.

FIG. 7F. A detailed view of the rail width selection feature of the first curtain bar of FIG. 7D, showing three alternative configurations.

FIG. 8A. A top sectional view of the first curtain bar attachment to a patient rail of a first width.

FIG. 8B. A top sectional view of the first curtain bar attachment to a patient rail of a second width.

FIG. 8C. A top sectional view of the first curtain bar attachment to a patient rail of a third width.

FIG. 8D. A perspective view of a mounting slot of the first curtain bar.

FIG. 8E. A rear view of the first curtain bar of FIG. 7A including one or more mounting slots.

FIG. 8F. A mounting bracket configured to attach the first curtain bar to a rail of a patient table.

FIG. 9A. An accessory rail configured to attach to the first curtain bar.

FIG. 9B. A detailed view of the accessory rail width selection feature of the accessory rail of FIG. 9A.

FIG. 9C. A top sectional view of the accessory rail attachment to the first curtain bar of a first width.

FIG. 9D. A top sectional view of the accessory rail attachment to the first curtain bar of a second width.

FIG. 9E. A top sectional view of the accessory rail attachment to the first curtain bar of a third width.

FIG. 10A. A perspective view of the anti-sag pivotable view joint.

FIG. 10B. A perspective view of an upper tab of the anti-sag pivotable joint.

FIG. 10C. A perspective view of a lower tab of the anti-sag pivotable joint.

FIG. 10D. A perspective view of another embodiment of the anti-sag pivotable view joint.

FIG. 10E. A perspective view of an upper tab of the anti-sag pivotable joint of FIG. 10D.

FIG. 11. Another embodiment of the shield assembly showing a first curtain bar, a second curtain bar, a third curtain bar, radiation blocking curtains, and radiation blocking panels.

FIG. 12. The shield assembly of FIG. 11, showing an exploded view of the components.

FIG. 13. A top perspective view of the shield assembly of FIG. 11, showing the rotation of a second curtain bar about one or more pivotable joints.

### DETAILED DESCRIPTION

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For example, the terms “approximately parallel” or “approximately vertical” refer to an angle within an acceptable degree of error or variation from true parallel or vertical, such as within 45, 25, 20, 15, 10, or 1° of true parallel or vertical. Numerical quantities given in this description are approximate unless stated otherwise, meaning that the term “about” or “approximately” can be inferred when not expressly stated. Claimed numerical quantities are exact unless stated otherwise.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “fastened”, or “coupled” to another feature or element, it can be directly connected, attached, fastened or coupled to the other feature or element or intervening features or elements may be present. In contrast, when a feature or element is referred to as being “directly connected”, “directly attached”, “directly fastened”, or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting. As used herein, the singular forms “a”, “an” and “the” are intended to include the plural forms as well (i.e., at least one of whatever the article modifies), unless the context clearly indicates otherwise.

Spatially relative terms, such as “under”, “below”, “lower”, “over”, “upper” and the like, may be used herein for ease of description to describe one element or feature's relationship to another when the apparatus is right side up as shown in the accompanying drawings.

Terms such as “at least one of A and B” should be understood to mean “only A, only B, or both A and B.” The same construction should be applied to longer list (e.g., “at least one of A, B, and C”). In contrast, terms such as “at least one A and at least one B” should be understood to require both A and B.

The terms “first”, “second”, “third,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The term “consisting essentially of” means that, in addition to the recited elements, what is claimed may also contain other elements (steps, structures, ingredients, components, etc.) that do not adversely affect the operability of what is claimed for its intended purpose as stated in this disclosure. This term excludes such other elements that adversely affect the operability of what is claimed for its intended purpose as stated in this disclosure, even if such other elements might enhance the operability of what is claimed for some other purpose.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

### B. Radiation Shield Assembly

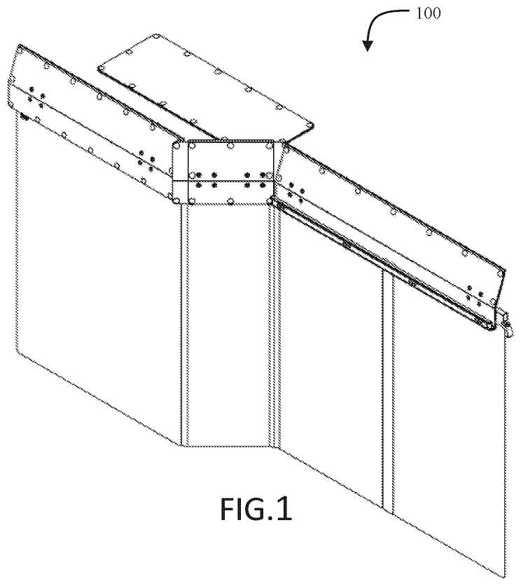

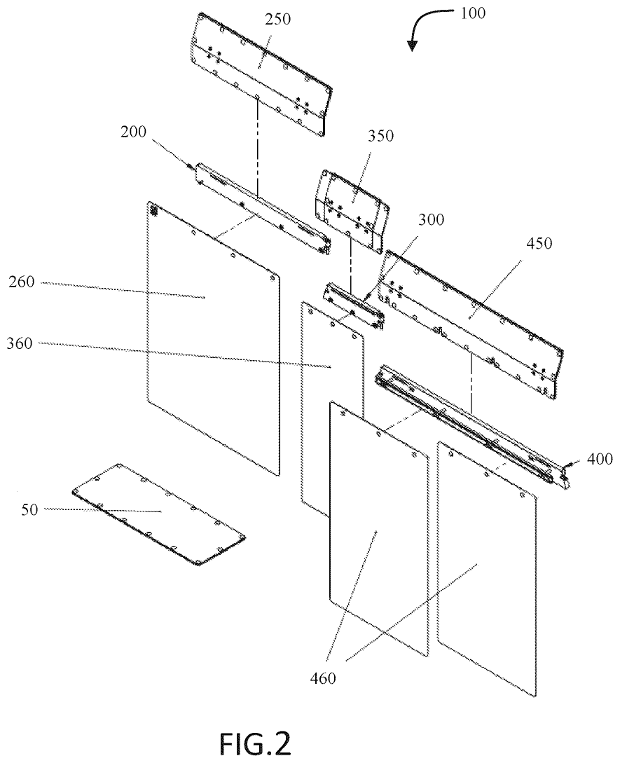

A radiation shield assembly 100 is provided, configured to block radiation emanating from a radiation source and supported by attachment to a rail of a patient table. As shown in FIGS. 1-2, the radiation shield assembly can include a first curtain bar assembly 400, second curtain bar assembly 300, and a third curtain bar assembly 200. The first curtain bar assembly 400 can attach to one or more radiation blocking curtains 460. Similarly, the second curtain bar assembly 300 can attach to a radiation blocking curtain 360, and first curtain bar assembly 200 can attach to a radiation blocking curtain 260. The radiation blocking curtains 260, 360, and 460 are configured to block radiation emanating from a radiation source below patient table (not shown). Radiation blocking curtains 260, 360, and 460 can be attached to the curtain bar assemblies 200, 300, and 400, respectively by fastening means such as screws, although any suitable fastening means can be implemented to attach the radiation blocking curtains to the curtain bars. Screws have the advantage of relatively high load capacity and ease of detachment and attachment. Also shown is a radiation blocking panel 450 that can be attached to the first curtain bar assembly 400, a radiation blocking panel 350 that can be attached to the second curtain bar assembly 300, and a radiation blocking panel 250 that can be attached to the third curtain bar assembly 300. Radiation blocking panel 250, 350, and 450 can be configured to block radiation emanating from a radiation source emanating from above a patient table. Also shown is an optional table mounted radiation shield 50, which is configured to attach directly to a standard size, rectangular cross section rail that runs along the side of the patient table (not shown). According to some embodiments, table mounted radiation shield 50 can be constructed of a flexible piece of material. Radiation curtains 260, 360, and 460 may be provided in the form of a flexible radiopaque material. Radiation curtains 260, 360, and 460 may be implemented as a shroud, a sleeve, and/or a curtain. They may be constructed from any suitable flexible and radiopaque material.

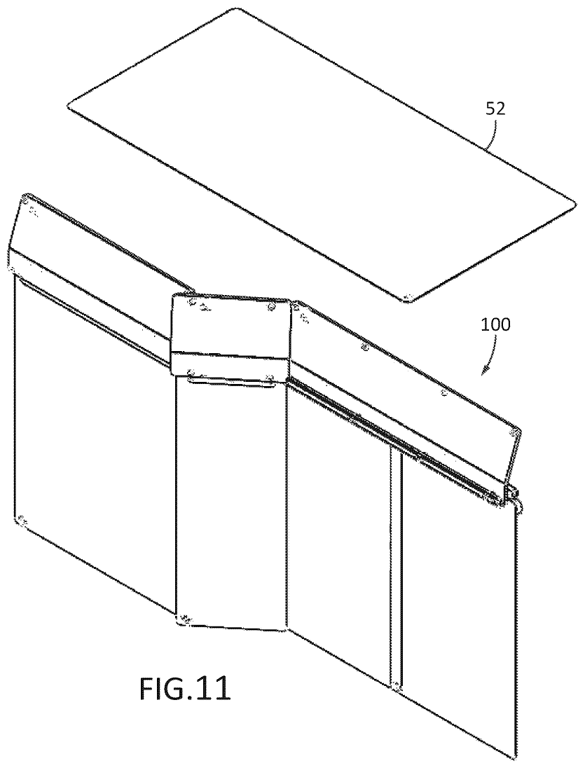

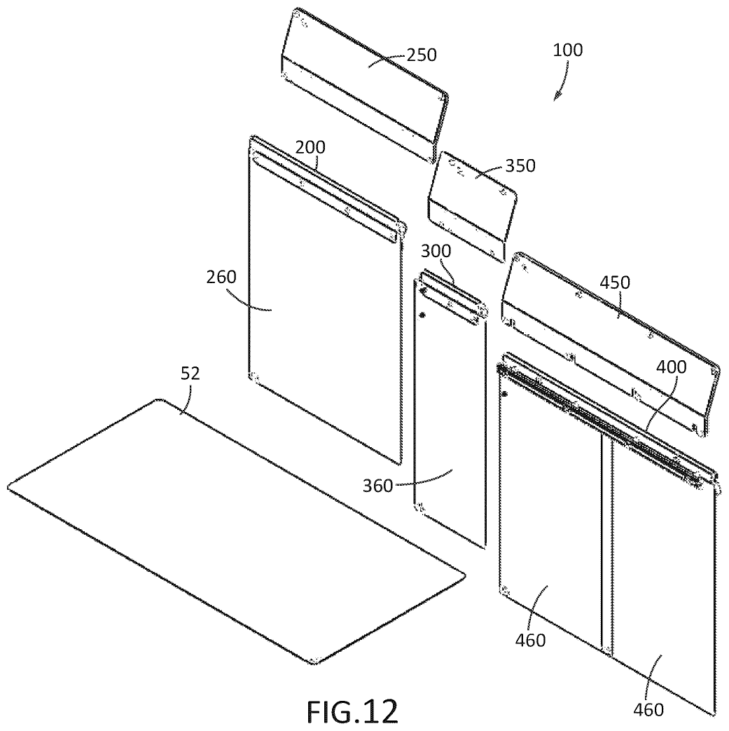

Another embodiment of radiation shield panel assembly 100 is shown in FIGS. 11-12. The radiation shield panel assembly 100 shown in FIG. 11 is similar to that radiation shield panel assembly 100 shown in FIG. 1, except that in place of optional table mounted radiation shield 50, a flexible radiation mat 52 can be used. Flexible radiation mat 52 is a multi-purpose radiation shielding material that can be used in conjunction with the radiation shield panel assembly 100 in situations where the procedure table shape warrants the need for supplemental protection. FIG. 12 shows an exploded perspective view of the radiation shield panel assembly 100 of FIG. 11. As shown, radiation shield assembly 100 shown in FIG. 12 is similar to that shown in FIG. 11, except that flexible radiation mat 52 is used in place of optional table mounted radiation shield 50.

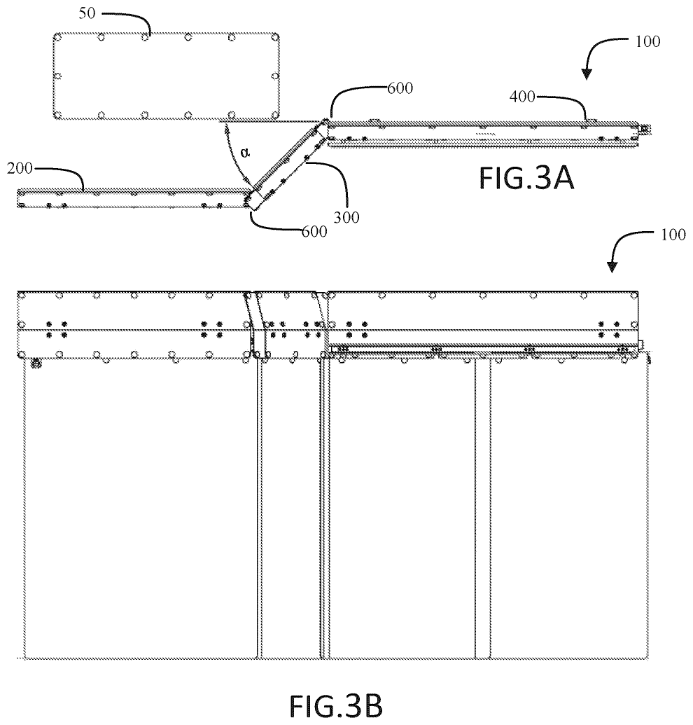

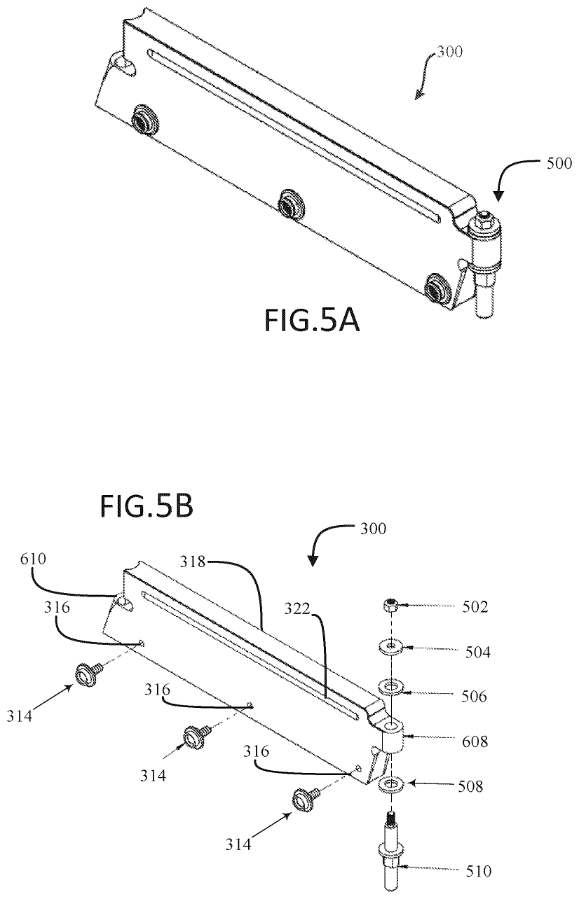

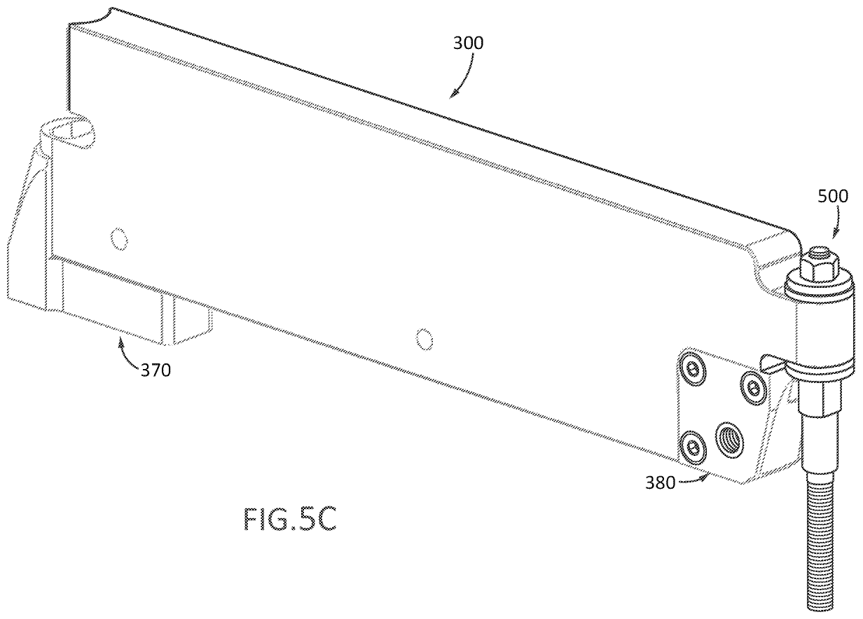

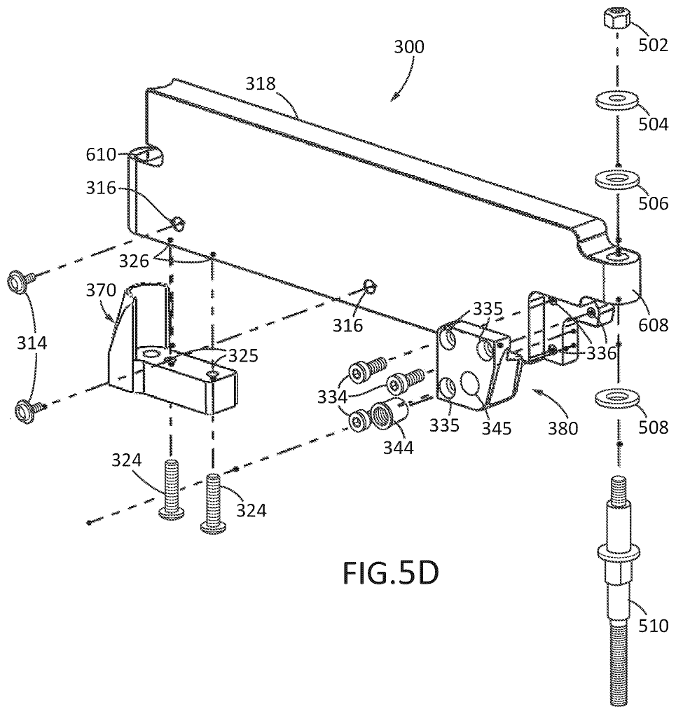

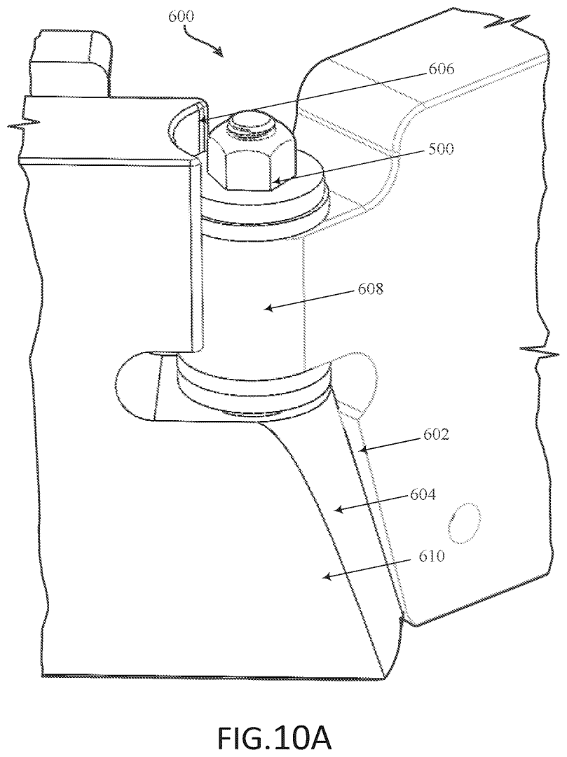

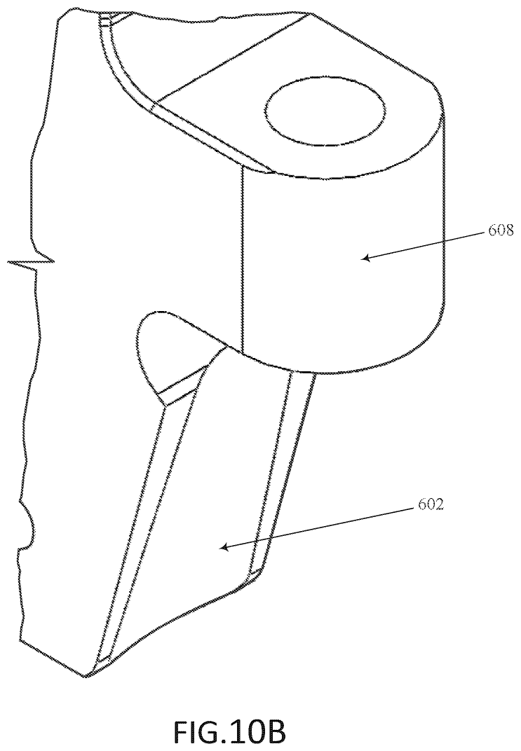

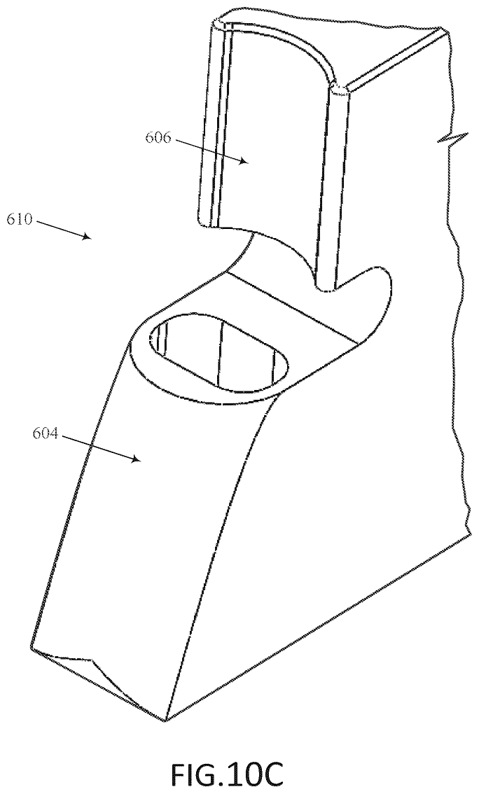

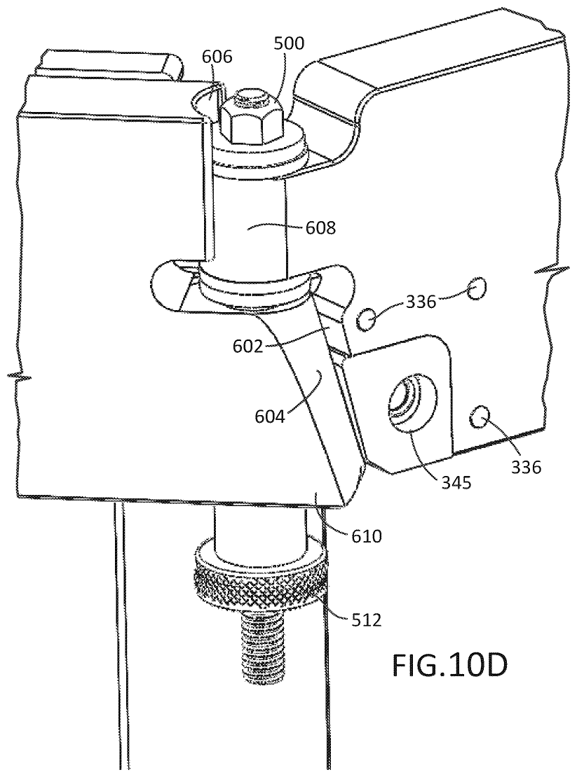

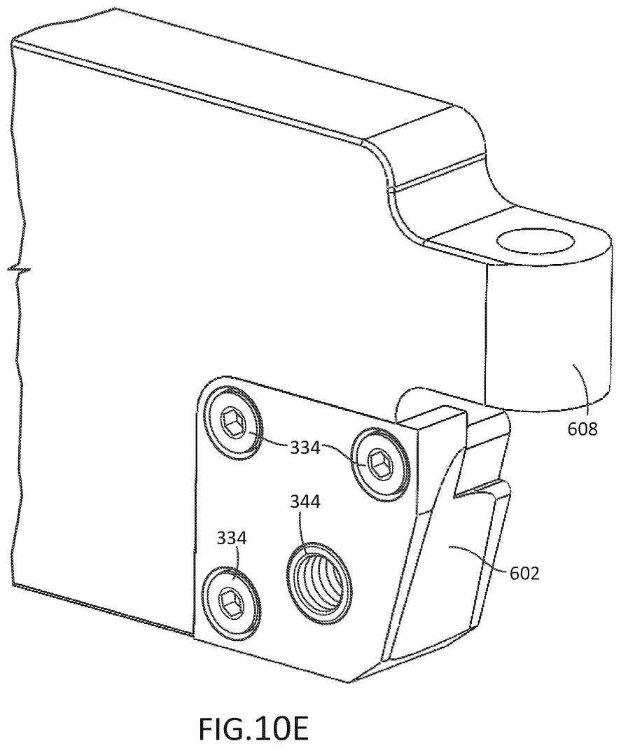

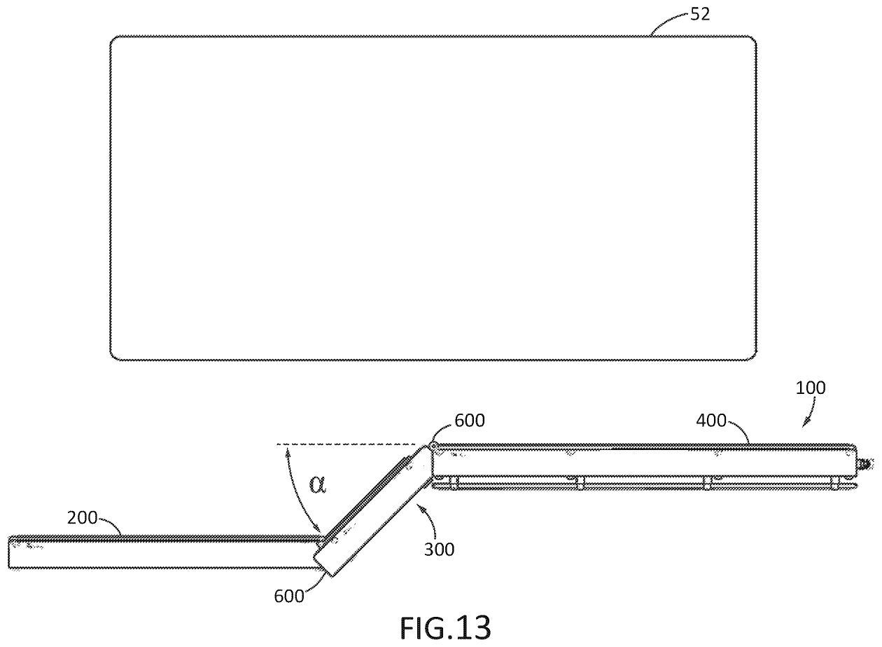

FIGS. 3A-3B show the radiation shield assembly 100 in which the second curtain bar assembly 300 is rotated with respect to the first curtain bar assembly 400, and the third curtain bar assembly 200 is rotated with respect to the second curtain bar assembly 300. On both ends of second curtain bar assembly 300 is an anti-sag pivotable joint 600. Anti-sag pivotable joints 600 allow the second curtain bar assembly 300 to pivot with respect to the first curtain bar assembly 400. By allowing the second curtain bar assembly 300 to pivot as shown, the third curtain bar assembly 200 can be swung out relative to the patient table, thereby moving the radiation shielding as desired by the user of the radiation shield assembly 100. Notably, the pivotable joint 600 is shown with an angle of rotation a relative to the first curtain bar assembly 400. In some embodiments, the pivotable joint 600 can rotate at an angle α (a “swing” angle) between 0 degrees and approximately 90 degrees. In some embodiments, the pivotable joint 600 can rotate at an angle α between 0 degrees and approximately 45 degrees. Similarly, the third curtain bar assembly 200 can also be configured to swing relative to the end of the second curtain bar assembly 300 at an angle α, which can be between approximately 0 degrees and approximately 90 degrees. In some embodiments, angle α can be between approximately 0 degrees and approximately 45 degrees. It should be noted that each anti-sag pivotable joint 600 can have a predetermined amount of resistance to motion, such that the second curtain rail assembly 300 and third curtain rail assembly 200 do not move during a procedure without input from the user of radiation shield assembly 100. The predetermined amount of resistance can be set by tightening a hinge bolt (described in more detail with respect to FIGS. 5A-5B) to a predetermined torque specification. In some embodiments, the predetermined torque to achieve the desired resistance of anti-sag pivotable joints 600 is between approximately 2 Nm and approximately 5 Nm, although in other embodiments other torque specifications can be used to fasten the hinge bolt through the anti-sag pivotable joints 600. As will be described in more detail with respect to FIGS. 10A-10C, each anti-sag pivotable joint 600 can be configured to prevent the second curtain rail assembly 300 and the third curtain rail assembly 200 from sagging with respect to the patient table. FIG. 13 shows another embodiment of the radiation shield assembly 100 of FIG. 3A. In contrast to the radiation shield assembly 100 of FIG. 3A, the embodiment shown in FIG. 13 includes a flexible radiation shield mat 52 in place of optional table mounted radiation shield 50.

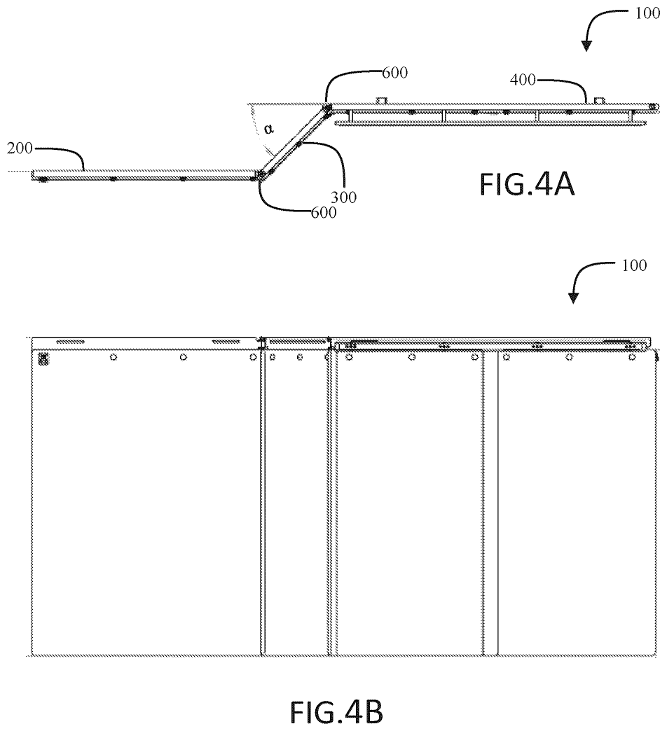

FIGS. 4A-4B show the first curtain bar assembly 400, second curtain bar assembly 300, and third curtain bar assembly 200 with the second curtain bar 300 rotated with respect to the first curtain bar 400, but without the radiation blocking panels 450, 350, and 250, attached to the first curtain bar assembly 400, second curtain bar assembly 300, and third curtain bar assembly 200, respectively. Although radiation shield assembly 100 is shown with three curtain bar assemblies 200, 300, and 400, radiation shield assembly can include more or fewer curtain bars. In some examples, radiation shield assembly 100 includes a single curtain bar (e.g., first curtain bar assembly 400). In other examples, radiation shield assembly 100 includes two curtain bars (e.g., first curtain bar assembly 400 and second curtain bar assembly 300). In other examples, radiation shield assembly includes three curtain bars (e.g., first curtain bar assembly 400, second curtain bar assembly 300, and third curtain bar assembly 200). In yet other examples, radiation shield assembly 100 can include more than three curtain bars (e.g., first curtain bar assembly 400, second curtain bar assembly 300, third curtain bar assembly 200, and one or more additional curtain bar assemblies).

FIGS. 5A-5B show second curtain bar assembly 300 in more detail. The second curtain bar assembly 300 includes the curtain bar body 318. Curtain bar body 318 may include an optional panel mounting slot 322 for fixedly attaching one or more radiation blocking panels (e.g., radiation blocking panel 350). Radiation panels 350 can be attached to the optional panel mounting slot via clips that snap into place within optional panel mounting slot 322. As shown, second curtain bar 300 can include one or more radiation blocking curtain attachment means 314 that are attached (e.g., threaded) into respective apertures 316. Radiation blocking curtain attachment means 314 are shown as snaps, but various other attachment means are envisioned such as screws, hook and loop, hooks, attachment via studs and a corresponding grommet, etc. On the left-hand side of curtain rail body 318 is a lower tab 610 that in conjunction with a complementary upper tab on an adjacent curtain bar assembly and a hinge bolt 500 assembly forms an anti-sag pivotable joint 600. The right hand side of curtain rail body 318 includes an upper tab 608 and shows how hinge bolt assembly 500 can be attached through the upper tab 608. As shown, hinge bolt assembly 500 includes an upper locknut 502, a washer 504, and a washer 506 that are mounted above the upper tab 608 and a washer 508 and pivot pin 510 below the upper tab 608. When fully assembled, pivot pin would pass through a lower tab (such as tab 610), with washer 508 being placed between the upper tab 608 and a corresponding lower tab. While washer 504 can be constructed of metal, washers 506 and 508 can be made from lubricant-filled nylon or plastic to provide lubrication to the hinge bolt assembly 500.

FIGS. 5C-5D show another embodiment of second curtain bar assembly 300 in more detail. In contrast to second curtain bar assembly 300 shown in FIGS. 5A-5B, the embodiment shown in FIGS. 5C-5D has detachable lower anti-sag joint insert 370 and detachable upper anti-sag joint insert 380. The exploded perspective of FIG. 5D shows how detachable lower anti-sag joint insert 370 and detachable upper anti-sag joint insert 380 are configured to attach to the rail body 318 of second curtain bar assembly 300. In contrast to the embodiment shown in FIGS. 5A-5B, second curtain bar assembly 300 shown in FIGS. 5C-5D lacks an optional panel mounting slot 322. Additionally, detachable lower anti-sag joint insert 370 and detachable upper anti-sag joint insert 380 are shown, which can be coupled to the rail body 318. Lower anti-sag joint insert 370 is attached to the rail body 318 via fasteners 324 which couple anti sag joint insert 370 and rail body 318 through apertures 325 of lower anti-sag joint insert 370 and apertures 326 of rail body 318. Similarly, upper anti-sag joint insert 380 is attached to the rail body 318 via fasteners 334 which couple anti sag joint insert 380 and rail body 318 through apertures 335 of upper anti-sag joint insert 380 and apertures 336 of rail body 318. In some embodiments, upper anti-sag joint inset 380 may additionally include an aperture 345 which allows for installation of an optional threaded insert 344 to be threaded through upper anti-sag joint insert 380. According to some embodiments, threaded insert 344 can serve as an insert that allows a radiation blocking curtain (e.g., one of radiation blocking curtains 260, 360, and 460) to be attached to the radiation shield assembly 100. In other words, curtain attachment means (e.g., curtain attachment means 214, 314, 414) can be configured to screw into the threads of threaded insert 344 so as to further secure a radiation blocking curtain to radiation shield assembly 100.

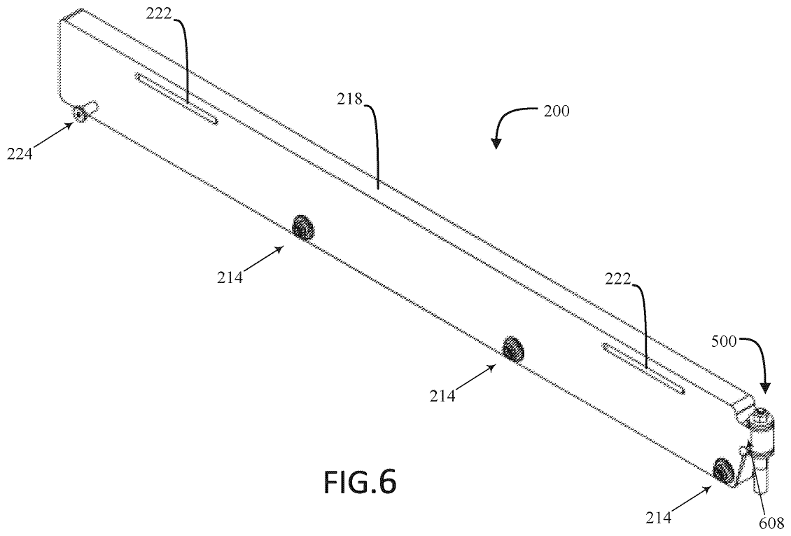

FIG. 6 shows a second curtain rail assembly 200 in more detail. As shown, second curtain rail assembly 200 can include a curtain rail body 218. Second curtain rail body 218 includes two optional panel mounting slots 222. Optional panel mounting slots 222 function similarly to optional panel mounting slot 322, in that one or more radiation shields (e.g., radiation shield 250) can be attached to the optional panel mounting slots 222 of curtain rail body 218 via clips that snap into place within optional panel mounting slot 222. Curtain rail body 218 also includes one or more radiation curtain attachment means 214, which are similar to the radiation curtain attachment means 314 as described with respect to FIGS. 5A-5B. Curtain attachment means 214 are configured to be attached (e.g., threaded) to the curtain rail body 218 through one or more apertures (not shown). On the right hand side of curtain rail body is a hinge bolt assembly 500 as previously described with respect to FIGS. 5A-5B mounted through an upper tab 608. Also included on the left hand side of curtain rail body 218 is an optional shoulder screw 224. Optional shoulder screw 224 may be used to attach the distal end of curtain 260 to securely fasten the distal end of curtain 260 to curtain rail body 218. The use of optional shoulder screw 224 over radiation curtain attachment means 214 may be useful to prevent curtain 260 from becoming inadvertently unfastened from the curtain rail body 218 during operation of the radiation shield assembly 100. However, in some embodiments, optional shoulder screw 224 can be replaced with radiation curtain attachment means 214.

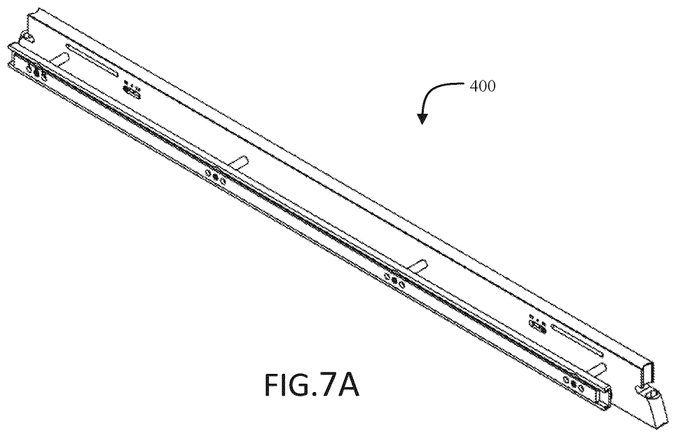

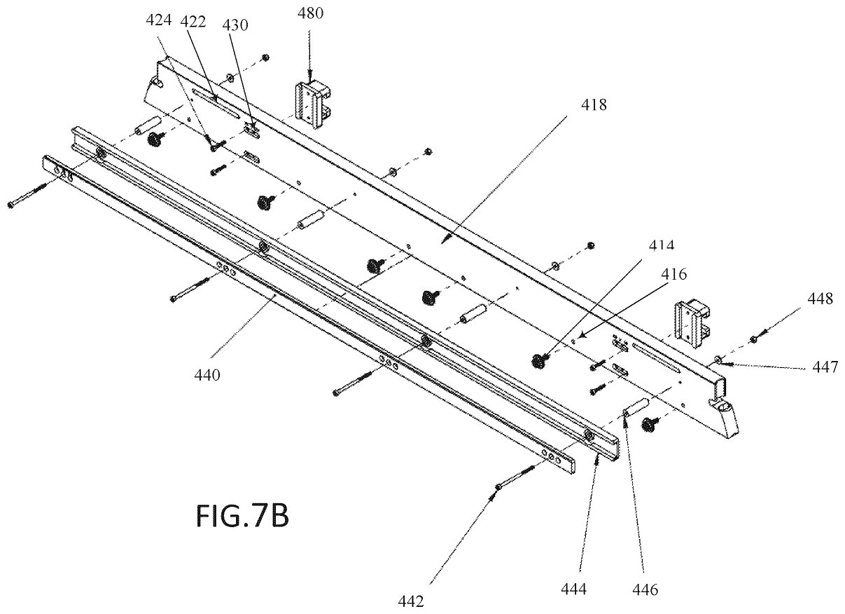

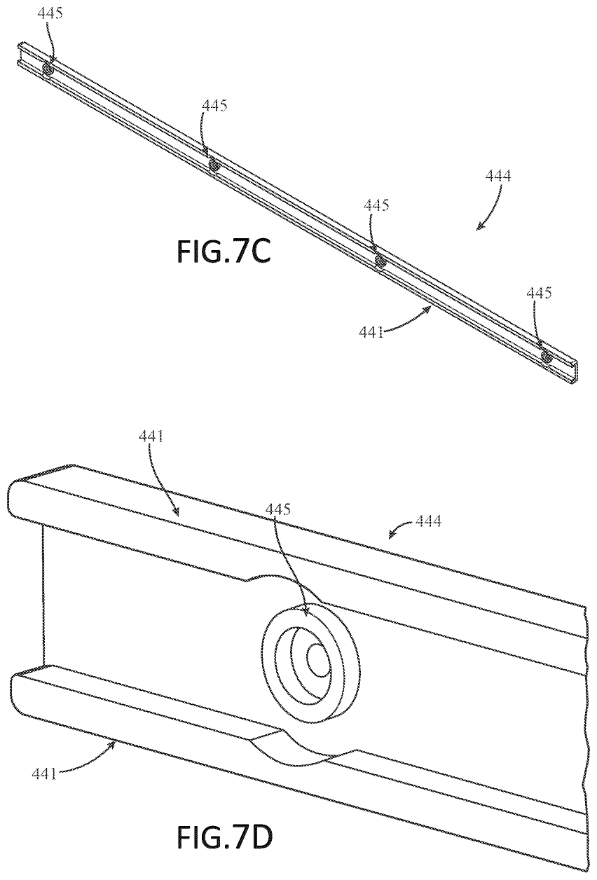

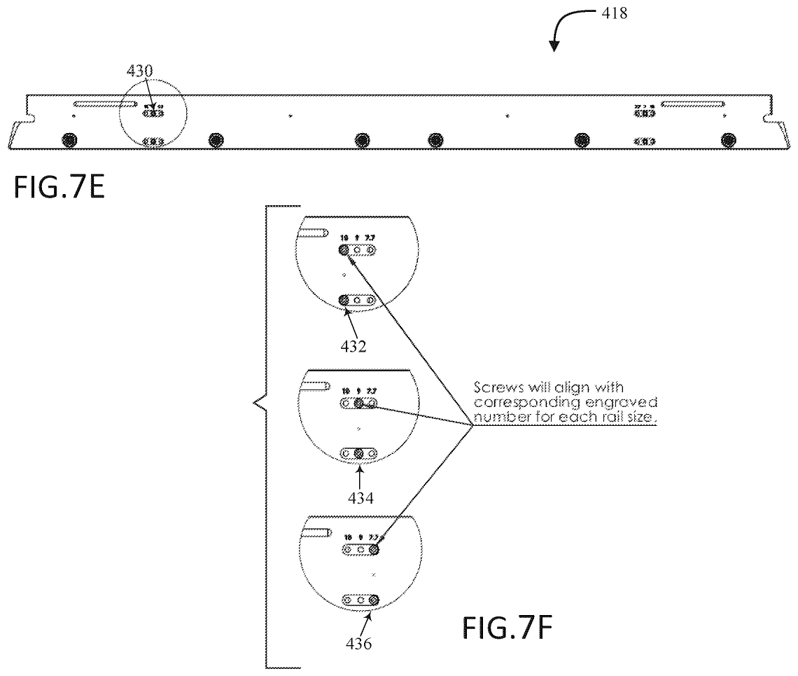

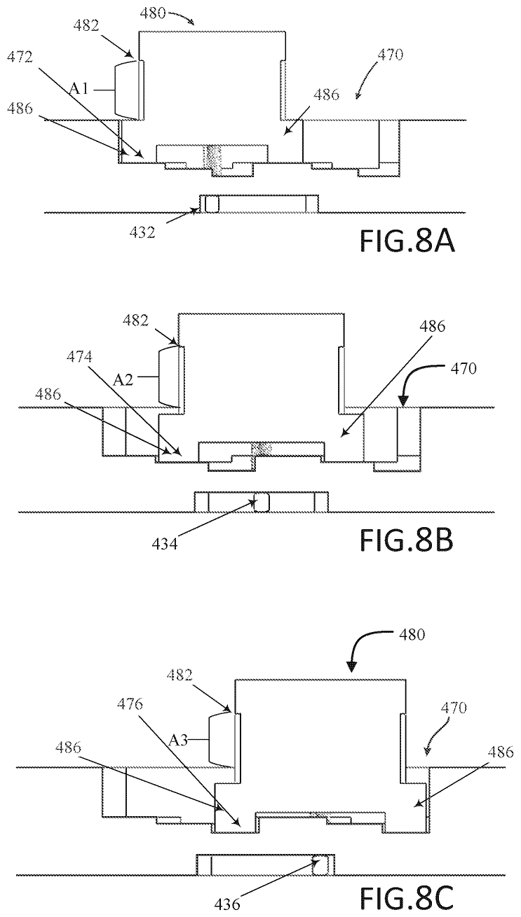

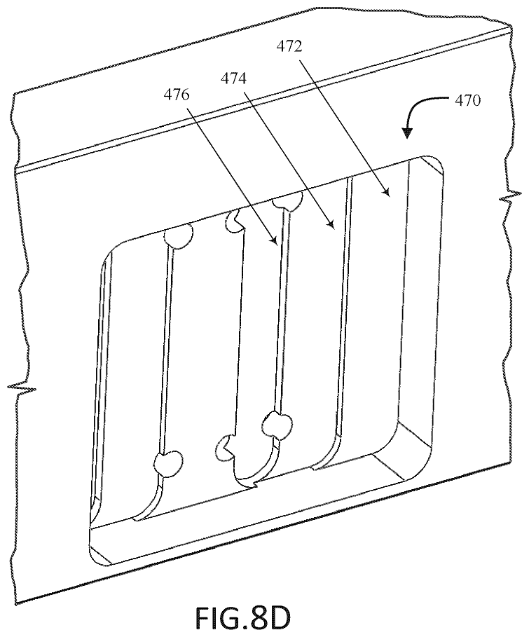

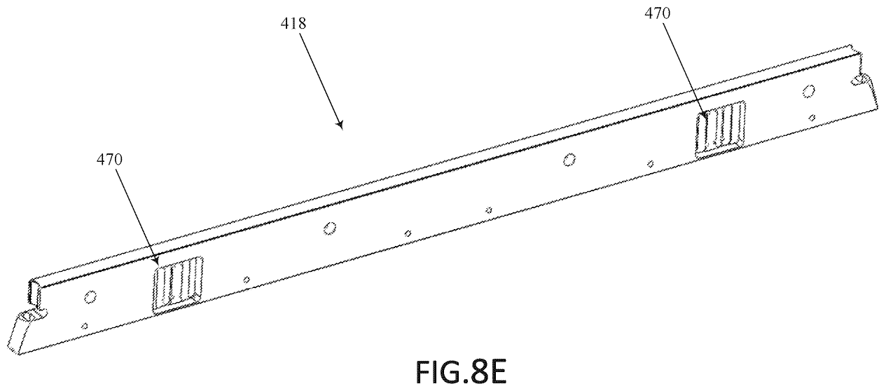

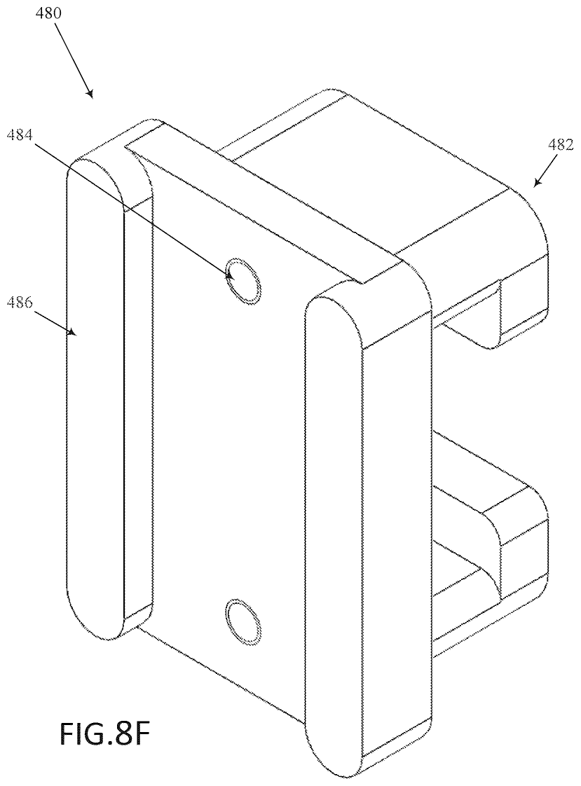

FIGS. 7A-7B show the first curtain rail assembly 400 in more detail. As shown, first curtain rail assembly 400 includes a curtain rail body 418. Curtain rail body 418 is configured to mount to a rail of a patient table by one or more rail mounting brackets 480. Rail mounting brackets 480 are configured to fit into complementary rail mounting slots on the back side of the curtain rail body 418, as shown and described in more detail below with respect to FIGS. 8A-8F. As will be described below, each rail mounting bracket 480 can include a C-shaped aperture configured to attach with the rail of a patient table. The C-shaped aperture can set to various widths to fit various widths of patient rails, making radiation shield assembly 100 universal to various standard patient table rail dimensions. As shown and described below in more detail with respect to FIGS. 8A-8F, the rail mounting brackets 480 are configured to selectively fit into a furrow pairs of various depths of the complementary rail mounting slots to vary the width of the C-shaped aperture of rail mounting bracket 480. The furrow pairs into which the rail mounting bracket fits can be selected by inserting an attachment bolt 424 into a respective aperture of apertures 430. Curtain rail body 418 also includes one or more optional panel mounting slots 422, which can be used to attach one or more radiation shields (e.g., radiation shields 450) to the optional panel mounting slots 422 of curtain rail body 418 via clips that snap into place within mounting slot 422. Curtain rail body 418 includes one or more radiation blocking curtain attachment means 414, which are similar to those described with respect to curtain rail body 318 and curtain rail body 218. As shown, the radiation blocking curtain attachment means 414 can be attached (e.g., threaded) into complementary apertures 416 within the curtain rail body 418. An accessory rail 440 can be attached to the curtain rail body 418 with one or more accessory bolt 442 that is fitted through complementary apertures in the accessory rail bracket 444 before passing through spacer 446 and being attached to the curtain rail body 418 by a washer 447 and threaded locknut 448. As will be described below, the accessory rail 440 can be set to various widths to accept medical devices that are configured to attach to different width rails.

FIGS. 7C-7D shows the accessory rail bracket 444 in more detail. As shown, accessory rail bracket 444 can include a plurality of accessory bolt apertures 445 that are designed to receive respective accessory bolts 442 to attach accessory rail 440 to accessory rail bracket 444 and curtain rail body 418 as described above. Additionally, accessory rail bracket 444 includes upper and lower lips 441 that are configured to hold accessory rail 440 to the accessory rail bracket 444.

FIGS. 7E-7F show the apertures 430 in more detail. A user of the assembly can select a respective aperture from apertures 430 through which to insert a pair of attachment bolts 424. For example, attachment bolt 424 can be inserted through aperture 432 to select a first width for the C-shaped aperture 482 of rail mounting bracket 480. Attachment bolt 424 can be inserted through aperture 434 to select a second width for the C-shaped aperture 482 of rail mounting bracket 480. Likewise, attachment bolt 424 can be inserted through aperture 436 to select a third width of the C-shaped aperture 482 of rail mounting bracket 480.

FIGS. 8A-8C show the positioning of the rail mounting bracket 480 within a respective furrow pair of a rail mounting slot 470 based on a selection of aperture 432, 434, or 436. On the rear of the curtain rail 418 can be a pair of rail mounting slots 470. Each rail mounting slot can include a plurality of furrow pairs of various depths within the rail mounting slot 470. The rail mounting bracket can include a pair of projections that are configured to fit into a respective furrow pair of the rail mounting slot 470, based on the selection of aperture 432, 434, and 436. For example, as shown in FIG. 8A, a user inserts attachment bolt 424 into aperture 432, and attachment bolt is threaded into threaded apertures 484 (shown in FIG. 8F). This positions the projections 486 of rail mounting bracket 480 into the shallowest furrow pairs 472. Shallowest furrow pairs 472 place the C-shape aperture 482 such that C-shape aperture has a width A1 configured to fit a patient table rail of width A1. In some examples, the width A1 can be approximately 10 mm to fit a standard 10 mm size patient rail.

As shown in FIG. 8B, the user inserts attachment bolt 424 into aperture 434, and attachment bolt is threaded into threaded apertures 484 (shown in FIG. 8F). This positions the projections 486 of rail mounting bracket 480 into the deeper furrow pairs 474. Deeper furrow pairs 474 place the C-shape aperture 482 such that C-shape aperture has a width A2 configured to fit a patient table rail of width A2. In some examples, the width A2 can be approximately 9 mm to fit a standard 9 mm size patient rail.

As shown in FIG. 8C, the user inserts attachment bolt 424 into aperture 436, and attachment bolt is threaded into threaded apertures 484 (shown in FIG. 8F). This positions the projections 486 of rail mounting bracket 480 into the deepest furrow pairs 476. Deepest furrow pairs 476 place the C-shape aperture 482 such that C-shape aperture has a width A3 configured to fit a patient table rail of width A3. In some examples, the width A3 can be approximately 7.7 mm to fit a standard 7.7 mm size patient rail.

FIG. 8D shows a rail mounting slot 470 found on the rear of curtain rail body 418. As shown, the rail mounting slot 470 includes a plurality of furrow pairs, including a shallow furrow pair 472, a deeper furrow pair 474, and a deepest furrow pair 476. Although rail mounting bracket 470 is shown with three pairs of furrow pairs, it should be understood that rail mounting bracket 470 can include any number of furrow pairs of distinct depths such that the width of the C-shaped aperture 482 can be varied to fit a variety of distinct rail widths of a patient table. FIG. 8E shows a rear perspective view of curtain rail body 418. As shown, curtain rail body 418 includes two rail mounting slots 470, although more or fewer rail mounting slots 470 can be included on curtain rail body 418.

FIG. 8F shows a perspective view of rail mounting bracket 480. As shown rail mounting bracket includes threaded apertures 484 into which a pair of attachment bolts 424 can be threaded (e.g., through one of apertures 432, 434, or 436). Also shown are projections 486 that are configured to fit into one of the respective pairs of furrows (e.g., furrow pairs 472, 474, or 476) to vary the width of the C-shaped aperture 482.

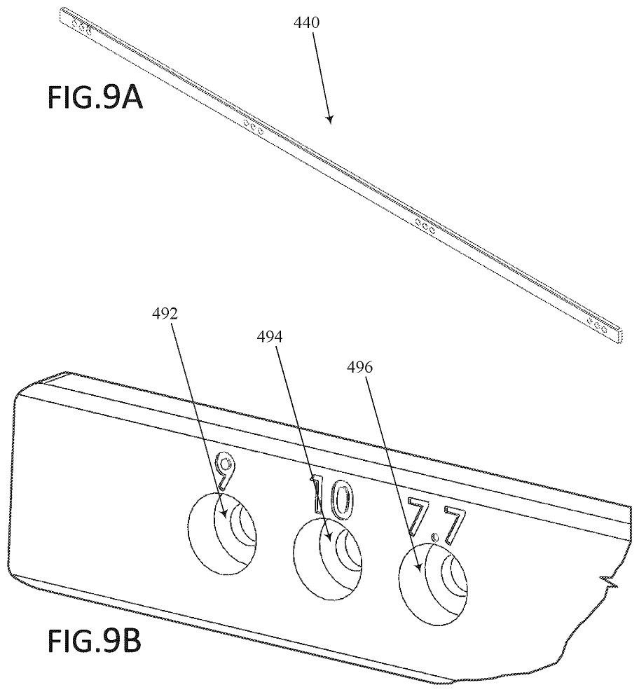

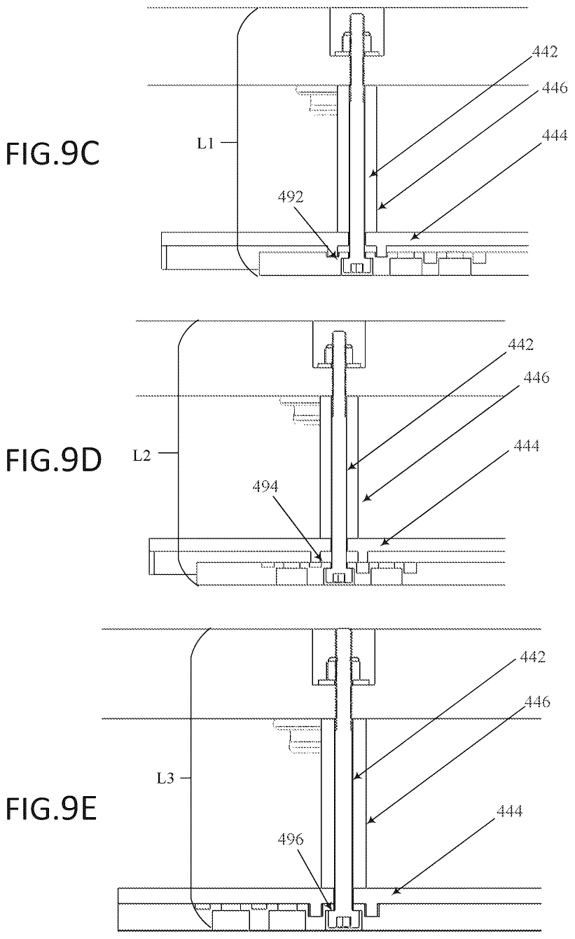

FIGS. 9A-9B show the accessory rail 440. As shown accessory rail 440 can include a plurality of apertures 492, 494, and 496. Each aperture has a distinct depth, such that the accessory rail 440 can be adjusted to various widths to fit a variety of medical devices. In the embodiment shown, aperture 492 is configured to give accessory rail 440 a width of approximately 9 mm such that medical devices configured to attach to a 9 mm width rail can be attached to the accessory rail 440. Aperture 494 is configured to give accessory rail 440 a width of approximately 10 mm such that medical devices configured to attach to a 10 mm width rail and be attached to the accessory rail 440. Aperture 496 is configured to give the accessory rail 440 a width of approximately 7.7 mm such that medical devices configured to attach to a 7.7 mm width rail and be attached to the accessory rail 440.

FIGS. 9C-9E show how the accessory rail 440 can be adjusted to various widths L1-L3. As shown, when an accessory bolt 442 is inserted through aperture 492, through accessory bracket 444, and spacer 446, the accessory rail can be adjusted to have a width L1. In this configuration, the width L1 of accessory rail 440 is approximately 9 mm. When an accessory bolt 442 is inserted through aperture 494, through accessory bracket 444, and spacer 446, the accessory rail can be adjusted to have a width L2. In this configuration, the width L2 of accessory rail 440 is approximately 10 mm. When an accessory bolt 442 is inserted through aperture 496, through accessory bracket 444, and spacer 446, the accessory rail can be adjusted to have a width L3. In this configuration, the width L3 of accessory rail 440 is approximately 7.7 mm. Although accessory rail 440 is shown to be adjustable to three distinct widths, it should be understood that accessory rail 440 can include any apertures of distinct depths such that the width of the accessory rail 440 can be varied to fit a variety of medical devices configured to fit different rail widths.

FIGS. 10A-10E show various aspects of the anti-sag pivotable joint 600. More specifically, FIG. 10A shows the anti-sag pivotable joint 600 in an assembled configuration. The anti-sag pivotable joint 600 includes a hinge bolt assembly 500 that couples upper tab 608 to a lower tab 610. The lower tab includes a convex conical surface 604. The upper tab includes a complementary concave conical surface that tightly interfaces (e.g., via direct contact) with convex conical surface 604 of the lower tab 610. The anti-sag pivotable joint 600 also includes a cylindrical cutout 606 that allows the hinge bolt (and thus the upper tab 608) to freely spin without making contact with the cylindrical cutout 606. Because the concave conical surface 602 directly contacts the convex conical surface 604, the weight of curtain rails that are not directly attached to the rail of the patient table (e.g., second curtain rail assembly 300 and/or third curtain rail assembly 200) are fully supported by the curtain rail assembly 400 that is directly attached to the rail of the patient table. Thus, assembly 100 stays substantially level with the rail of the patient table. FIG. 10B shows the upper tab 608 and the complementary concave conical surface 602 and FIG. 10C shows the lower tab 610, which includes the convex conical surface 604 and the cylindrical cutout 606. FIG. 10D shows an alternative embodiment anti-sag pivotable joint 600 in an assembled configuration. The anti-sag pivotable joint 600 shown in FIG. 10D is similar to the one in FIG. 10A, except that detachable upper anti-sag joint insert 380 is shown in this example. In contrast to the embodiment shown in FIG. 10A, upper anti-sag joint insert 380 is attached to the rail body 318 via fasteners 324 that secure upper anti-sag joint insert 380 to rail body 318 via apertures 336. Optional threaded insert aperture 345 is shown, which allows the installation of optional threaded insert 344. Also shown is fastener 512 that can be used to tighten and secure hinge bolt assembly 500. According to some embodiments, once hinge bolt assembly is placed through the anti-sag pivotable joint 600, fastener 512 can be threaded on to prevent hinge bolt assembly 500 from being pulled out of anti-sag pivotable joint 600. FIG. 10E shows the embodiment of FIG. 10D, in which the upper anti-sag joint insert 380 is attached to the rail body 318 via one or more fasteners 334. Threaded insert 344 is shown installed within the upper anti-sag joint insert 380, allowing for a radiation blocking curtain to be secured thereto via a fastener (e.g., curtain attachment means 214, 314, 414, etc.).

### C. Conclusion

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

1-20. (canceled)

21. A radiation shield assembly for blocking radiation emanating from a radiation source positioned below a patient table, the radiation shield assembly comprising:
a curtain bar configured to support a radiation blocking curtain, wherein the curtain bar includes—
a first surface and a second surface opposite the first surface, and
a slot within the first surface, the slot including a first furrow having a first depth and a second furrow having a second depth different than the first depth; and
a bracket configured to secure the curtain bar to a rail of the patient table, wherein the bracket includes—
a first side having a first tab and a second tab, wherein the first tab and the second tab at least partially define an opening sized and shaped to receive the rail, and
a second side having a sizing feature sized and shaped to be positioned within either of the first furrow or the second furrow,
wherein positioning the sizing feature within the first furrow causes the first tab and the second tab to extend a first distance relative to the first surface of the curtain bar, and
wherein positioning the sizing feature within the second furrow causes the first tab and the second tab to extend a second distance relative to the first surface of the curtain bar, wherein the second distance is different than the first distance.

22. The radiation shield assembly of claim 21 wherein the sizing feature is a protrusion.

23. The radiation shield assembly of claim 21 wherein:
the first furrow is one of a pair of first furrows,
the second furrow is one of a pair of second furrows, and
the sizing feature is one of a pair of protrusions.

24. The radiation shield assembly of claim 21 wherein the opening is c-shaped.

25. The radiation shield assembly of claim 21 wherein the curtain bar further includes:
a first aperture in the slot, wherein the first aperture is configured to receive a fastener to couple the bracket to the curtain bar when the sizing feature is positioned within the first furrow; and
a second aperture in the slot, wherein the second aperture is configured to receive the fastener to couple the bracket to the curtain bar when the sizing feature is positioned within the second furrow.

26. The radiation shield assembly of claim 21 wherein the slot includes a third furrow having a third depth, and wherein positioning the sizing feature within the third furrow causes the first tab and the second tab to extend a third distance relative to the first surface of the curtain bar, and wherein the third distance is different than the first distance and the second distance.

27. The radiation shield assembly of claim 21, further comprising an accessory rail assembly configured to be secured to the curtain bar, wherein the accessory rail assembly is adjustable between two or more widths.

28. The radiation shield assembly of claim 27 wherein the accessory rail assembly includes an accessory rail and an accessory rail bracket having a track for slidably receiving the accessory rail.

29. The radiation shield assembly of claim 28 wherein the track includes an opening for receiving a fastener for coupling the accessory rail to the accessory rail bracket, and wherein the accessory rail includes:
a first aperture having a first depth such that, when the first aperture is aligned with the opening in the track, the accessory rail assembly has a first width, and
a second aperture having a second depth such that, when the second aperture is aligned with the opening in the track, the accessory rail assembly has a second width, different than the first width.

30. A radiation shield assembly for blocking radiation emanating from a radiation source positioned below a patient table, the radiation shield assembly comprising:
a curtain bar configured to support a radiation blocking curtain; and
a bracket configured to secure the curtain bar to a rail of the patient table,
wherein a relative position of the bracket relative to the curtain bar is adjustable such that the radiation shield assembly is configured to be coupled to different size rails on the patient table.

31. The radiation shield assembly of claim 30 wherein the curtain bar includes a slot having a plurality of different depths, and wherein the bracket is positionable within the slot at the plurality of different depths.

32. The radiation shield assembly of claim 30 wherein a width of the radiation shield assembly is adjustable.

33. A radiation shield assembly for blocking radiation emanating from a radiation source positioned below a patient table, the radiation shield assembly comprising:
a first curtain bar configured to support a first radiation blocking curtain;
a second curtain bar configured to support a second radiation blocking curtain; and
an anti-sag joint pivotably coupling the second curtain bar to the first curtain bar,
wherein the first curtain bar is configured to be attached to a rail of the patient table and the second curtain bar is configured to be cantilevered and supported by the anti-sag joint.

34. The radiation shield assembly of claim 33 wherein the anti-sag joint includes:
a first tab having a convex conical surface, and
a second tab having a concave conical surface sized and shaped to receive the convex conical surface,
wherein at least one of the convex conical surface or concave conical surface is configured to rotate relative to the other of the concave conical surface or the convex conical surface.

35. The radiation shield assembly of claim 34 wherein the convex conical surface is configured to directly support the concave conical surface.

36. The radiation shield assembly of claim 34 wherein the first curtain bar includes the first tab, and wherein the second curtain bar includes the second tab.

37. The radiation shield assembly of claim 34, further comprising a bolt configured to extend at least partially through the first tab and the second tab.

38. The radiation shield assembly of claim 34 wherein the second tab includes a removable insert having the convex conical surface.

39. The radiation shield assembly of claim 33 wherein a resistance of pivoting the second curtain bar relative to the first curtain bar about the anti-sag joint is adjustable.

40. The radiation shield assembly of claim 33 wherein the anti-sag joint is a first anti-sag joint, and wherein the radiation shield assembly further comprises:
a third curtain bar configured to support a third radiation blocking curtain; and
a second anti-sag joint pivotably coupling the third curtain bar to the second curtain bar,
wherein the third curtain bar is further configured to be cantilevered and supported by the second anti-sag joint.
